(19) United States
(12) Patent Application Publication
(10) Pub. No.: US 2024/0390300 A1
(43) Pub. Date: Nov. 28, 2024

(54) ADVANCED METHOD AND KIT FOR WOUND TREATMENT

(71) Applicant: JOHNSON & JOHNSON CONSUMER INC., Skillman, NJ (US)

(72) Inventors: Fernanda M. B. Bruzadin, Skillman, NJ (US); Jenny Du-Soriano, New Hope, PA (US); Gabriella John, Hamilton, NJ (US); Caihua Cecilia Wang, Kendall Park, NJ (US)

(21) Appl. No.: 18/322,135

(22) Filed: May 23, 2023

(51) Int. Cl. A61K 31/14; A61F 13/02; A61K 9/06; A61K 31/167; A61K 38/12; A61K 38/14

(52) U.S. Cl. CPC A61K 31/14; A61F 13/0206; A61K 9/06; A61K 31/167; A61K 38/12; A61K 38/14

(57) ABSTRACT

The present invention relates to advanced methods of treating a wound comprising the steps of cleansing the wound with an antiseptic formulation, applying an antibiotic formulation to the wound, covering the wound with an adhesive bandage, and repeating these steps for at least two additional, consecutive days.

### FIELD OF THE INVENTION

The present invention relates generally to advanced methods for treating wounds. The invention also relates to a kit comprising associated treatment components and instructions for their effective use.

### BACKGROUND OF THE INVENTION

The treatment of minor wounds, cuts and abrasions on the skin has included the use of various washing or cleansing formulations, antibiotic formulations, and bandages. See, e.g., BAND-AID® Brand Value Pack including a container of first aid antiseptic pain relieving liquid, a container of first aid antibiotic ointment, and a box of adhesive bandages with a three-step process: 1. Clean, 2. Treat, 3. Protect.

However, there is no specific guidance on how to potentially use these treatment components in combination to reduce the time for wound healing. Nor is there specific guidance on steps following an initial treatment, e.g., after the first application of antiseptic and antibiotic. Indeed, controversy exists regarding the efficacy of using antiseptic formulations to treat wounds. For example, a 1987 study indicated that repeated antiseptic treatment of contaminated blister wounds rated poorest in mean ranking of overall clinical appearance and wound-healing rate of wounds. Leyden, J. J., MD, and Bartelt, N. M., Comparison of Topical Antibiotic Ointments, a Wound Protectant, and Antiseptics for the Treatment of Human Blister Wounds Contaminated with Staphylococcus aureus, The Journal of Family Practice, Vol. 24., No. 6:601-604 (1987).

While cleansing & covering a wound is always recommended, few sources recommend including an antiseptic cleanser, especially on the wound, and even then, sources instruct “antiseptics should be used to disinfect only intact skin surrounding the wound safter the removal of all organic matter.” Bernard, D. B. (2020). Chapter 41: Minor Burns, Sunburns, and Wounds. In Krinsky, D. L., Ferreri, S. P., and Hemstreet, B., et al., Handbook of nonprescription drugs: An interactive approach to self-care (19th ed.). Washington, DC: American Pharmacists Association. doi: https://doi-org.jerome.stjohns.edu/10.21019/9781582122656.ch41.

Indeed, some clinicians have indicated that repeated use of antiseptic formulations may be detrimental to the healing process. For example, Atiyeh et al. state, wound cleansers may affect normal human cells and may be antimitotic adversely affecting normal tissue repair. Repeated and excessive treatment of wounds with antiseptics without proper indications may have negative outcomes or promote a microenvironment similar to those found in chronic wounds.” Atiyeh, B. S., Dibo, S. A., and Hayek, S. N., Wound cleansing, topical antiseptic and wound healing, Int. Wound J 2009; 6:420-430). In addition, Atiyeh et al. reference a prior study, “a [recently] published experimental study about the effectiveness of different methods currently used for the irrigation of open wounds comparing normal saline solution, bacitracin solution, castile soap and benzalkonium chloride with use of a pulsatile lavage device (19 psi) then using the same animal model to compare bulb syringe and pulsatile lavage irrigation with saline solution.” The authors concluded, “approaches used to remove bacteria from wounds, such as irrigants other than saline solution or high-pressure devices, may not have the best clinical outcome,” citing Owens, B. D., White, D. W., and Wenke, J. C., Comparison of irrigation solutions and devices in a contaminated musculoskeletal wound survival model, J. Bone Joint Surg. Am. 2009; 91:92-8).

Therefore, what is needed is a more effective wound care process that does not hinder healing by cleaning, treating, and protecting the wounds when there is a risk of infection.

### SUMMARY OF THE INVENTION

Further objects and advantages of the invention will be readily apparent to those skilled in the art from the following detailed description, taken in conjunction with the sheets of drawings.

Surprisingly, in one aspect of the present invention, we have found that an advanced method of treating a wound including the steps of cleansing the wound with an antiseptic formulation, applying an antibiotic formulation to the wound, covering the wound with an adhesive bandage, and repeating these steps for at least two consecutive days for at least two consecutive days results in improved wound healing with no evidence of damage to the skin or delayed healing from repeated applications of antiseptic.

### BRIEF DESCRIPTION OF THE DRAWINGS

Embodiments of this invention will now be described in greater detail, by way of illustration only, with reference to the accompanying drawings, in which:

FIG. 1 depicts the steps of an advanced method for wound treatment; wherein, an antiseptic formulation, an antibiotic formulation and an adhesive bandage are applied to a wound in a stepwise fashion for at least two consecutive days.

FIG. 2 depicts the steps of an alternative advanced method for wound treatment; wherein an antiseptic formulation is applied to the wound, an antibiotic formulation is applied to an adhesive bandage, and then the adhesive bandage containing the antibiotic formulation is applied to the wound with said steps being completed for at least two consecutive days.

### DETAILED DESCRIPTION OF THE INVENTION

The present invention relates to advanced methods for treating wounds that have been shown clinically to improve the wound healing process.

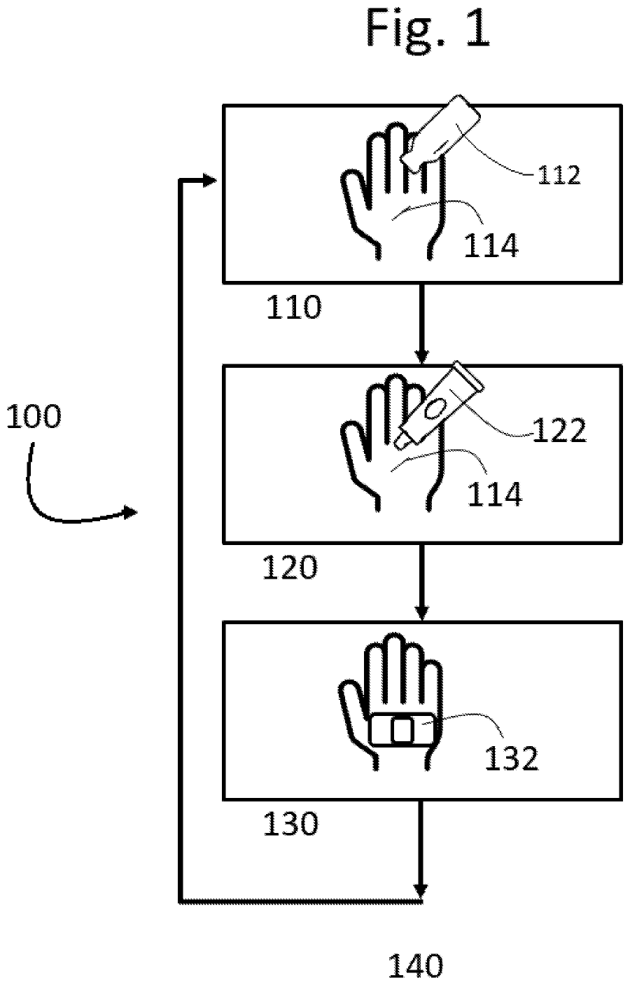

FIG. 1 illustrates a first embodiment of an advanced method for treating wounds of the present invention. This advanced method for wound treatment 100 comprises the stepwise application of three treatment components: an antiseptic formulation, an antibiotic formulation, and an adhesive bandage. Thus, in a first step 110, antiseptic formulation 112 is applied to a wound 114, e.g., on a wounded hand 116. In a second step 120, an antibiotic formulation 122 is applied to the wound 114. In a third step 130, adhesive bandage 132 is applied to cover the wound 114. After one day, the original adhesive bandage 132 is removed, and steps 110, 120 and 130 (covering the wound with a clean adhesive bandage 132′) are repeated as indicated in box 140. The repeated steps indicated in box 140 may be repeated on the 3rd day and consecutive days thereafter up to about 7 days or such a time when wound 114 is judged to be sufficiently healed.

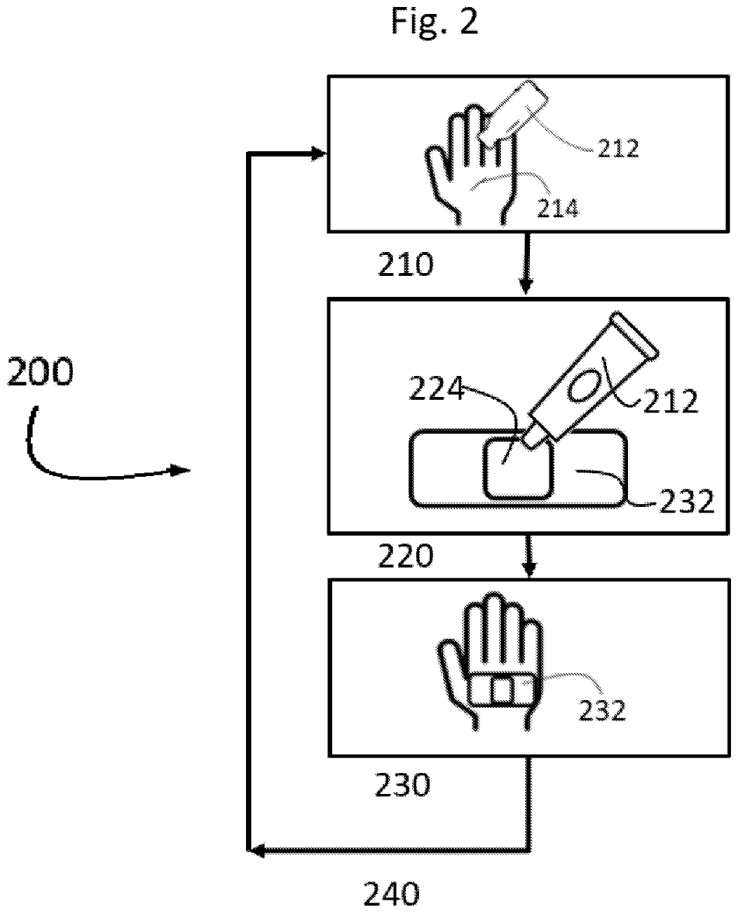

FIG. 2 illustrates a second embodiment of an advanced method for treating wounds of the present invention. Advanced method for wound treatment 200 also comprises the stepwise application of three treatment components: an antiseptic formulation 210, an antibiotic formulation 220, and an adhesive bandage 230. Thus, in a first step 210, an antiseptic formulation 212 is applied to a wound 214, e.g., on a wounded hand 216. In a second step 220, an antibiotic formulation 222 is first applied to the pad 224 of an adhesive bandage 232. In a third step 230, the adhesive bandage 232 containing the antibiotic formulation 222 is applied to cover the wound 214 such that the antibiotic formulation 212 contacts wound 214. After one day, the original adhesive bandage 232 is removed, and steps 210, 220 and 230 (covering the wound with a clean adhesive bandage 132′ dosed with the antibiotic formulation 222) are repeated as depicted in box 240. The repeated steps indicated in box 240 may be repeated on the 3rd day and consecutive days thereafter up to about 7 days or such a time when wound 214 is judged to be sufficiently healed.

The antiseptic formulation 112, 212 of this invention may be any over-the-counter antiseptic formulations used by consumers. Antiseptics are biocidal products that can kill or impact the growth of disease-causing bacteria, fungi or viruses in, or on, living tissue, e.g., on the skin ideally without damaging healthy tissue or being absorbed into systemic circulation. Preferred antiseptic formulations are liquids that may be directly applied to the wound, e.g., by pouring, squirting, spraying, including as an aerosol or foam to limit manual contact with the wound. Alternatively, the liquid antiseptic formulation may be applied to a carrier such as a sterile cloth or wipe before application to a wound, e.g., as a prepackaged antiseptic formulation-impregnated wipe. For example, a small amount of the liquid antiseptic formulation may be directly squirted onto wound (without touching wound) and subsequently allowed to air dry.

Antiseptic formulations may contain one or more active ingredients including ethyl alcohol, isopropyl alcohol or benzalkonium chloride. Preferably, the antiseptic formulation comprises benzalkonium chloride, a monograph ingredient. Water may be used as the solvent for the antiseptic active ingredient. The preferred concentration of benzalkonium chloride in the antiseptic formulation is about 0.01 to 5% w/w. A benzalkonium chloride concentration of 0.05 to 1% w/w is more preferred. An analgesic capable of imparting pain relief may also be advantageously incorporated in the antiseptic formulation. Lidocaine hydrocholoride is a particularly preferred analgesic. The preferred concentration of lidocaine hydrochloride in the antiseptic formulation is about 0.05 to 5% w/w. A lidocaine hydrochloride concentration of 1 to 3% w/w is more preferred. Additional auxiliary agents to increase efficacy and tolerance may be added to the antiseptic formulation including aloe barbadensis leaf juice, disodium ethylenediaminetetraacetic acid (disodium EDTA), poloxamer 188, sodium chloride and sodium citrate. In some embodiments, the wound is washed conventionally using soap and water or other liquid skin cleansers prior to application of the antiseptic formulation.

The antibiotic formulation 122, 222 of this invention may be any over-the-counter antiseptic formulations used by consumers. Antibiotic agents are those chemical compounds that kill bacteria which may be present on the surface of the skin. In certain embodiments, suitable antibiotic agents may include polymyxin B sulfate, bacitracin zinc, and/or neomycin sulfate. An antibiotic formulation comprising a mixture of polymyxin B sulfate, bacitracin zinc and neomycin sulfate is particularly preferred. Such antibiotic formulations may come in the form of liquids, ointments, creams, gels, or aerosols which may be applied directly to the wound from the product container. In some embodiments, the antibiotic agent is formulated within an ointment base comprising petrolatum. For example, a small amount (an amount equal to the surface area of the tip of a finger) of the antibiotic formulation may be transferred from a clean, gloved finger to the wound. Alternatively, a small amount of the antibiotic formulation may be applied to the absorbent pad of an adhesive bandage (as described below) for transfer to the wound.

The preferred concentration of the antibiotic agent in the antibiotic formulation is 0.5 to 5% w/w. A concentration of the antibiotic agent in the antibiotic formulation of 1-3% w/w is more preferred. A local anesthetic capable of numbing the skin and minimizing pain may also be advantageously incorporated in the antibiotic formulation. Pramoxine hydrocholoride is a particularly preferred local anesthetic. The preferred concentration of pramoxine hydrochloride in the antibiotic formulation is about 0.1 to 5% w/w. A pramoxine hydrochloride concentration of 0.5 to 3% w/w is more preferred. Additional auxiliary agents to increase stability and efficacy may be added to the antibiotic formulation including butylated hydroxytoluene (BHT), tocopherol and ethylenediaminetetraacetic acid (EDTA).

The adhesive bandages 132, 232 of this invention may be any adhesive bandages available to consumers for the purpose of self-care. Generally, an adhesive bandage is a small, flexible sheet of material which is adhesive on one side and non-adhesive on the other. A smaller, non-adhesive, absorbent pad is typically attached to the adhesive side to absorb wound exudate. The flexible sheet of material may be liquid and air permeable, and in a preferred embodiment, the flexible sheet of material is liquid impermeable to more fully isolate the wound. The absorbent pad is placed so it covers the wound, and the surrounding edges of adhesive material are extended and compressed so they adhere to the skin around the wound. The adhesive side of the bandage is typically covered by a removable release liner to facilitate packaging by the manufacturer and handling by the consumer. Adhesive bandages are generally packaged individually and fully enclosed in a sealed wrapper. A plurality of individually-wrapped adhesive bandages are generally supplied in a box or carton containing about 2 to about 100 individually-wrapped adhesive bandages. Adhesive bandages may be comprised of woven fabric or polymeric films such as polyolefin (e.g., polyethylene) polyvinylchloride (PVC), polyester, latex or polyurethane films. The absorbent pad of the adhesive bandage may comprise cotton or other natural fibers, polymeric fibers, hydrogel or hydrocolloid materials, or the like. The adhesive used on the adhesive bandage may be a tacky, biocompatible substance such as acrylic polymers and the like.

The present invention will be better understood from a consideration of the following illustrative examples.

### EXAMPLES

The examples that follow are based on a single center, randomized, comparator-controlled, 16-day clinical trial which compared healing rates and infection protection for different treatment regimens including an antiseptic formulation, an antibiotic formulation, and an adhesive bandage. A total of 34 subjects completed study participation, with all 34 subjects included in the intent-to-treat (ITT) population. The subjects enrolled in this trial were 25- to 55-year-old males and females of Fitzpatrick skin types I-III who had uniform skin color on both volar forearms, and who had consented to participate in this clinical trial. At Screening (Visit 1; 3 to 7 days prior to Baseline), subjects were provided with an auxiliary cleanser to use on their forearms and for all body cleansing in place of their regular body cleanser for the duration of the study. At Baseline or Day 0 (Visit 2), a Sciton Er: YAG 2940 laser was used to induce six partial-thickness (i.e. minor) wounds on the subjects' forearms (three per arm). The wounds created by this method heal by the migration of epidermal cells from the dermal appendages located in the wound's base (dermal islands) and/or wound borders, and mimic minor wounds similar to real life scraped skin, typically healing in less than 16 days if left untreated. Each wound test site for each subject was randomly assigned to one of the following:

| Example | Treatment description |
|---|---|
| Comparative | Uncovered wound observed through 7 days. |
| Example A | |
| Comparative | Adhesive bandage alone applied initially on |
| Example B | Day 0 and on each of 6 days thereafter. |
| Comparative | Antibiotic formulation applied initially on Day |
| Example C | 0 and on each of 6 days thereafter. |
| Example 1 | Antiseptic formulation + antibiotic formulation + |
| | adhesive bandage applied on Day 0 and on |
| | each of 2 days thereafter. Left uncovered and |
| | untreated from Day 3 through Day 7. |
| Example 2 | Antiseptic formulation + antibiotic formulation + |
| | adhesive bandage applied on Day 0 and on |
| | each of 6 days thereafter. |

The treatment components used in each of the examples are further described below:

TABLE 1
Antiseptic formulation

| | Amount |
|---|---|
| Component | (weight %) |
| Water | 96.01069 |
| Lidocaine HCl | 2.00000 |
| Poloxamer 188 | 1.00000 |
| Sodium Chloride | 0.61000 |
| Disodium EDTA | 0.20000 |
| Benzalkonium Chloride | 0.14300 |
| Sodium Citrate | 0.02631 |
| Aloe Barbadensis Leaf Juice | 0.01000 |
| Total | 100.00000 |

TABLE 2
Antibiotic formulation

| | Amount |
|---|---|
| Component | (weight %) |
| Petrolatum | 97.5800 |
| Pramoxine HCl | 1.0000 |
| Bacitracin Zinc | 0.7200 |
| Neomycin sulfate | 0.5800 |
| Polymyxin B Sulfate | 0.1200 |
| Total | 100.0000 |

### Adhesive Bandage

Standard of care bandage comprising a flexible fabric material and polyurethane film and an absorbent pad.

### Wound Assessment

Each wound site was photographed (through Day 7) and assessed at specified intervals by clinical grading of wound healing parameters. The primary endpoint of this study was the Composite Healing Score, which was calculated from the clinical grading of wound healing parameters as follows:

Composite Healing Score=[general wound appearance score+smoothness score+epithelial confluence score]−[erythema score+edema score+crusting/scabbing score]

The composite healing score on a 25-point scale (−12 thru+12) is indicative of the extent of wound healing and was calculated for each wound site at each evaluation day. The greater the composite healing score, the great the extent of wound healing. Composite healing score was summarized at each time point and was analyzed within-treatment and between-treatment. The within-treatment comparison was performed at each post-baseline time point by comparing the post-baseline scores with the baseline score (defined as the post-wound score on Day 0) within each treatment using the paired t-test. The between-treatment comparison was performed by comparing the change from baseline (defined as post-baseline score minus baseline score) between treatments using a mixed effect analysis of covariance (ANCOVA) model.

The secondary endpoints were analyzed in a similar way as for the composite healing score and were as follows:
Clinical Grading of Wound Healing—Erythema
Clinical Grading of Wound Healing—Edema
Clinical Grading of Wound Healing—General Wound Appearance
Clinical Grading of Wound Healing—Smoothness
Clinical Grading of Wound Healing—Epithelial Confluence
Clinical Grading of Wound Healing—Crusting/Scabbing

TABLE 3
Composite Healing Scores (extent of healing increases with increasing values)

| | | | | Example 1 | Example 2 |
|---|---|---|---|---|---|
| | | | | Antiseptic | Antiseptic |
| | | | | Formulation + | Formulation + |
| | | Comparative | Comparative | Antibiotic | Antibiotic |
| | | Example B | Example C | Formulation + | Formulation + |
| | Comparative | Adhesive | Antibiotic | Adhesive | Adhesive |
| | Example A | Bandage | Formulation | Bandage | Bandage |
| | Untreated | Alone | Alone | (3 days) | (7 days) |
| Day 1 | −3.51 | −2.10 | −3.59 | −1.35 | −1.26 |
| Day 2 | −3.93 | −3.03 | −4.03 | −1.84 | −1.84 |
| Day 3 | −4.03 | −2.91 | −3.79 | −1.72 | −1.84 |
| Day 4 | −4.05 | −3.23 | −3.47 | −1.62 | −1.30 |
| Day 5 | −3.01 | −2.26 | −2.84 | −0.50 | −0.01 |
| Day 6 | −1.71 | −1.15 | −1.94 | 0.59 | 0.99 |
| Day 7 | −0.31 | −0.01 | −0.56 | 1.68 | 1.90 |

The composite healing scores of the wounds were first determined on the first day after wounding (Day 1) before the treatments were repeated as prescribed. Even after 1 day, it was evident that the composite healing scores of Examples 1 and 2 were significantly higher than those of Comparative Examples A, B & C indicating a greater extent of wound healing in Examples 1 & 2 versus that in Comparative Examples A, B & C. On Day 2, the composite healing scores decreased in all cases due to the formation of scabs and redness as expected; however, the composite healing scores of Examples 1 and 2 were still significantly greater than those in Comparative Examples A, B & C. On Day 3, the composite healing score of untreated wounds (Comparative Example A) further decreased; whereas, the composite healing score stabilized or slightly increased in the remaining examples. Again on Day 3, the composite healing scores of Examples 1 and 2 were still significantly greater than those in Comparative Examples A, B & C. On Days 4 through 7, the composite healing scores significantly increased each day in Examples 1 and 2 and remained consistently higher on each day when compared to the scores in Comparative Examples A, B & C. Taken as a whole, these data demonstrate that the daily stepwise application of an antiseptic formulation, an antibiotic formulation and an adhesive bandage result in a greater extent of healing when compared to the use of an antibiotic formulation or adhesive bandage alone or when no treatment is used at all. Further, no evidence of delayed healing or skin necrosis is present, even with the repeated application of antiseptic to the wound (not just the surrounding intact skin tissue).

What is claimed is:

1. A method of treating a wound comprising the steps of:
a. cleansing the wound with an antiseptic formulation;
b. applying an antibiotic formulation to the wound;
c. covering the wound with an adhesive bandage; and
d. repeating steps a)-c) for at least two additional, consecutive days.

2. The method of claim 1 wherein steps a)-c) are repeated for at least three additional, consecutive days.

3. The method of claim 1 wherein steps a)-c) are repeated for at least four additional, consecutive days.

4. The method of claim 1 wherein the antiseptic formulation is a liquid and the step of cleansing the wound comprises directly applying the liquid antiseptic formulation to the wound.

5. The method of claim 1 wherein the antiseptic formulation is a liquid and the step of cleansing the wound further comprises applying the liquid antiseptic formulation to a carrier and then cleansing the wound with said antiseptic bearing carrier.

6. The method of claim 1 further comprising the step of washing the wound and surrounding skin tissue with soap and water or liquid skin cleanser prior to steps a) through c).

7. The method of claim 1 wherein the antiseptic formulation comprises an antiseptic active ingredient and an analgesic.

8. The method of claim 7 wherein the antiseptic active ingredient comprises benzalkonium chloride and the analgesic comprises lidocaine hydrochloride.

9. The method of claim 1 wherein the antiseptic formulation comprises benzalkonium chloride.

10. The method of claim 1 wherein the antibiotic formulation is an ointment.

11. The method of claim 1 wherein the antibiotic formulation comprises polymyxin B sulfate, bacitracin zinc, neomycin sulfate or mixtures thereof.

12. The method of claim 1 wherein the antibiotic formulation comprises an antibiotic agent and a local anesthetic.

13. The method of claim 12 wherein the antibiotic agent comprises polymyxin B sulfate, bacitracin zinc, neomycin sulfate or mixtures thereof and the local anesthetic comprises pramoxine hydrochloride.

14. The method of claim 1 wherein the antibiotic formulation is applied to the adhesive bandage prior to covering the wound with the adhesive bandage.

15. The method of claim 1 wherein the adhesive bandage comprises a flexible sheet of material having an absorbent pad and an adhesive disposed on a first surface thereof.

16. The method of claim 15 wherein the flexible sheet of material is liquid impermeable.

17. A method of treating a wound consisting essentially of the steps of:
a. cleansing the wound with an antiseptic formulation comprising benzalkonium chloride;
b. applying an antibiotic formulation comprising at least one antibiotic to the wound;
c. covering the wound with an adhesive bandage; and
d. repeating steps a)-c) for at least two additional, consecutive days.

18. A method of treating a wound consisting of the steps of:
a. cleansing the wound with an antiseptic formulation comprising benzalkonium chloride;
b. applying an antibiotic formulation comprising at least one antibiotic to the wound;
c. covering the wound with an adhesive bandage; and
d. repeating steps a)-c) for at least two additional, consecutive days.
